(19) United States
(12) Patent Application Publication
(10) Pub. No.: US 2006/0154980 A1
(43) Pub. Date: Jul. 13, 2006

(54) IMPROVED COX-2 INHIBITORY COMPOSITIONS AND THERAPEUTIC REGIMEN

(76) Inventor: John Westcott Finley, Lansdale, PA (US)

Correspondence Address: THADDIUS J. CARVIS, 102 NORTH KING STREET, LEESBURG, VA, 20176, US

(21) Appl. No.: 11/275,493

(22) Filed: Jan. 10, 2006

Related U.S. Application Data
(60) Provisional application No. 60/642,933, filed on Jan. 11, 2005.

(51) Int. Cl. A61K 31/353; A61K 31/557; A61K 31/202; A61K 31/05; A61K 31/12

(52) U.S. Cl. 514/454; 514/573; 514/560; 514/685; 514/733

(57) ABSTRACT

This invention provides improved COX-2 inhibitory compositions comprising a combination of a selective inhibitor of COX-2 and a prostacyclin promoter, especially an antioxidant of the type known as food extracts. The invention also provides a regimen treating a disease that is responsive to inhibition of cyclooxygenase-2, comprising administering to a patient suffering from such a disease the combination of the invention comprising a selective inhibitor of COX-2 and a prostacyclin promoter in amounts and at intervals effective to therapeutically inhibit COX-2 while maintaining a therapeutically significant level of prostacyclin production.

### RELATED APPLICATION

This application claims priority to U.S. Provisional Patent Application No. 60/642,933 filed Jan. 11, 2005, the disclosure of which is incorporated herein by reference.

### BACKGROUND OF THE INVENTION

The invention relates to improvements in COX-2 inhibitory compositions and regimens utilizing them.

In recent years, several pharmaceutical compositions for treatment of cyclooxygenase-2 (COX-2) mediated diseases have been developed. These compositions have enjoyed wide acceptance among physicians and patients, alike, due to their ability to target COX-2, an enzyme implicated in inflammation, while largely avoiding COX-1, which is believed to protect the stomach from gastric acids. This class of drugs, then, differs from other anti-inflammatories which had targeted both. Among these are the drugs which are identified in the U.S. FDA Orange Book under the proprietary names, VIOXX, CELEBREX and BEXTRA. In addition there are similar drugs approved in the United Kingdom, under the proprietary names PREXIGE and ARCOXIA.

Recently, however, the manufacturer of VIOXX withdrew that drug from the market after a pattern surfaced halfway through a 3-year colon polyp prevention study. Heart attacks and strokes had occurred at a higher rate among the approximately 1300 volunteers on VIOXX (3.5%) than among a like group taking a placebo (1.9%). Concern has now spread to other COX-2 inhibitors, and there is no clear answer for those seeking relative certainty.

COX-2 inhibitors target COX-2 with varying degrees of specificity. COX-2 is an enzyme implicated in inflammation, and is inhibited while COX-1, which protects the stomach from gastric acids, is largely avoided. The COX-2 inhibitors are believed to be involved with altering the mechanisms for the conversion of fatty acids to prostacyclin (prostaglandin [PG] I₂) and thromboxane. Of these, prostacyclin is believed to play a role in arresting platelet formation and preventing cell clumping. It is also related to dilating blood vessels. The other, thromboxane, works differently by encouraging platelet clumping and constricting vessels. Other anti-inflammatory drugs, like naproxen, which are nonspecific to COX-2 inhibition, suppress both prostacyclin, which plays a role in inflammation and thromboxane. This can result in stomach problems for patients taking them over long periods of time. COX-2 inhibitors, on the other hand, block only prostacyclin formation. The blocking of prostacyclin to too great an extent could eliminate its essential function in arresting platelet formation and preventing cell clumping and/or blood vessel dilation.

There is a need for improvements in COX-2 inhibitory compositions and regimens utilizing them, to enable them to alleviate the problems that specific COX-2 inhibition may be connected with, while avoiding the problems associated with stomach acid irritation of the stomach tissue.

### SUMMARY OF THE INVENTION

It is an object of the invention to provide an improved class of COX-2 inhibitory compositions.

It is another object of the invention to provide an improved regimen for treatment of a patient having a disease that is responsive to inhibition of cyclooxygenase-2.

It is an object of a preferred aspect of the invention to provide a composition and treatment regimen for inhibiting COX-2 production and positively promoting generation of prostacyclin in mammals, particularly humans and domestic pets.

These and other objects are accomplished by the invention, which provides compositions and treatment regimens.

In one aspect the invention provides improved COX-2 inhibitory compositions comprising a combination of a selective inhibitor of COX-2 and a prostacyclin promoter, especially an antioxidant of the type known as food extracts.

In another of its aspects the invention provides a regimen treating a disease that is responsive to inhibition of cyclooxygenase-2, comprising administering to a patient suffering from such a disease the combination of the invention comprising a selective inhibitor of COX-2 and a prostacyclin promoter in amounts and at intervals effective to therapeutically inhibit COX-2 while maintaining a therapeutically significant level of prostacyclin production.

Preferred aspects of the invention will be described below.

### DESCRIPTION OF THE INVENTION

The invention is intended to provide therapeutic relief to mammalian subjects, especially humans, but also other treatable mammals including dogs, cats, horses, goats, sheep, cattle, and other domesticated animals and pets. The group that can benefit from the invention includes those suffering from a disease that is responsive to inhibition of cyclooxygenase-2. It is an advantage of the invention that the included compositions and regimens effectively inhibit COX-2 production while positively promoting generation of prostacyclin in mammals.

Among the pharmaceutical compositions for treatment of cyclooxygenase-2 (COX-2) mediated diseases are any of those having the ability to target COX-2, an enzyme implicated in inflammation, while largely avoiding COX-1, which is believed to protect the stomach from gastric acids. This class of drugs differs from other anti-inflammatories which target both. Among these are the drugs which are identified in the U.S. FDA Orange Book under the proprietary names, VIOXX (rofecoxib, see U.S. Pat. No. 5,474,995), CELEBREX (U.S. Pat. No. 5,466,823), BEXTRA (U.S. Pat. No. 5,633,272). Also included ARE drugs approved in the United Kingdom, under the proprietary names PREXIGE (Lumiracoxib) and ARCOXIA (etoricoxib). A more complete listing of COX-2 inhibitory compositions is found in U.S. Patent Publication No. 20040034085, which is hereby incorporated by reference including all pertinent references identified therein.

The group of compositions useful in the invention as promoting generation of prostacyclin in mammals include the antioxidants of the type known as food extracts particularly from foods (including beverages) rich in flavonoids and other antioxidant compounds of therapeutic significance. Among these therapeutically significant foods and derivatives are, as purees, juices and extracts from the group of berries, fruits, vegetables, legumes, oils, nuts, seeds and dried fruit, cereals, roots, spices, plant extracts and tubers. The following is a listing of foods rich in antioxidants by group:

Berries
Dog rose
Crowberry
Bilberry/wild
blueberry
Black currant
Sour cherry
Strawberry
Blueberry
Cranberry
Raspberry
Cloudberry
Fruit
Cherry
Black cherry
Pomegranate
Grape
Orange
Plum
Pineapple
Lemon
Dates
Kiwi
Clementine
Grapefruit
Legumes
Broad beans
Pinto beans
Ground nut
Soybeans
Nuts, seeds, and dried fruit
Walnuts
Sunflower seeds
Apricots
Prunes
Almonds
Chocolate
Phenolic
enriched
chocolate
Vanilla and
vanilla extracts
Oils
Soy oil and soy
oil distillates
Canola oil
canola oil
distillates
Corn Oil and
corn oil
distillates
Rice bran oil and
rice bran oil
distillates
Fish oil and
omega-3 rich
fractions from
fish oil
Omega-3 rich oil
form microalgae
Omega-3 fatty
acids and esters
Walnut oil
Flaxseed oil
Plant Extracts
Rosmarinic acid
Curcumin
Resveratrol
Pycnogenol
Apple skin extract
Grape seed extract
Grape skin extract
Spices
Rosemary
Oregano
Basil
Turmeric
Pepper
Peppermint
Spearmint
Vegetables
Kale
Chili pepper
Red cabbage
Peppers
Parsley
Artichoke
Brussels Sprouts
Spinach
Cereals
Barley
Millet
Oats
Corn
Wheat
Cereal bran
Cereal germ and
germ oil
Roots and Tubers
Ginger
Red Beets

The above flavonoid-rich foods are preferably processed by means known to the art to preserve and/or enrich the active flavonoid compounds. Those skilled in the art will recognize that the exact compositions having the desired prostacyclin promoting active ingredients have not been elucidated for most of these foods, but that the foods can be used in effective amounts to achieve the desired effects.

In vivo oxidation of lipids and the formation of free radicals are associated with inflammation. The addition of antioxidants to the system removes free radicals and may inhibit their formation, helping control negative outcomes associated with in vivo oxidation and inflammation. Another advantage of the invention relates to improved effectiveness in prevention of colon cancer without the risks associated with COX-2 inhibitors involved in the study that resulted in the withdrawal of VIOXX from the market.

This invention also provides a regimen for treating a disease that is responsive to inhibition of cyclooxygenase-2, comprising administering to a patient suffering from such a disease the combination of the invention comprising a selective inhibitor of COX-2 and a prostacyclin promoter in amounts and at intervals effective to therapeutically inhibit COX-2 while maintaining a therapeutically significant level of prostacyclin production. The preferred improved COX-2 inhibitory compositions comprise a combination of a selective inhibitor of COX-2 and as a prostacyclin promoter, an antioxidant rich in flavonoid compounds effective in promoting prostacyclin in mammals. The preferred regimen will provide the COX-2 inhibitory composition at its normally recommended dose, as can be determined, for example from the noted U.S. FDA Orange Book and the PDA, as desired. The effective level of the prostacyclin promoter will be determined by suitable assay, such as discussed for example by Polagruto, et al., J Med Food, 6 (4) 2003, 301-308, to show a positive response of significant therapeutic effect.

Preferably, the amount of the prostacyclin promoter should be in excess of the amount shown by the assay, preferably from about 50 to 1000% excess, most preferably at a level of from about 100 to 500% excess. In one specific example, extracted anti-inflammatory agents from plant tissue comprise one or more of curcumin, resveratrol, pycnogenol and rosmarinic acid, in an amount of from 100 mg to about 500 mg for each employed. Other extracted anti-inflammatory agents can be employed at proportional levels, with the objective being to provide at least sufficient activity to reduce apparent Cox-2 activity or measurable metabolic products 50%, as determined by urinary levels of PGEm or 8-iso-prostaglandin F2α.

The above description is for the purpose of teaching the person of ordinary skill in the art how to practice the invention. It is not intended to detail all of those obvious modifications and variations, which will become apparent to the skilled worker upon reading the description. It is intended, however, that all such obvious modifications and variations be included within the scope of the invention which is defined by the following claims. The claims are meant to cover the claimed components and steps in any sequence which is effective to meet the objectives there intended, unless the context specifically indicates the contrary.

1. Improved COX-2 inhibitory compositions comprising a combination of a selective inhibitor of COX-2 and a prostacyclin promoter.

2. COX-2 inhibitory compositions according to claim 1, wherein the prostacyclin promoter comprises an antioxidant of the type known as food extracts.

3. A composition according to claim 2, wherein the prostacyclin promoter comprises one or more of curcumin, resveratrol, pycnogenol and rosmarinic acid.

4. A regimen for treating a disease that is responsive to inhibition of cyclooxygenase-2, comprising administering to a patient suffering from such a disease the combination of the invention comprising a selective inhibitor of COX-2 and a prostacyclin promoter in amounts and at intervals effective to therapeutically inhibit COX-2 while maintaining a therapeutically significant level of prostacyclin production.

5. A regimen for treating a disease that is responsive to inhibition of cyclooxygenase-2, comprising administering to a patient suffering from such a disease the combination of the invention comprising a selective inhibitor of COX-2 and a source of omega-3 fatty acid to serve as a substrate for prostacyclin formation, the amounts and intervals of administration being effective to therapeutically benefit the patient.

6. A regimen for treating a disease that is responsive to inhibition of cyclooxygenase-2, comprising administering to a patient suffering from such a disease the combination of the invention comprising a selective inhibitor of COX-2 with extracted anti-inflammatory agents from plant tissue, the amounts and intervals of administration being effective to therapeutically benefit the patient.

7. A regimen according to claim 6 wherein the extracted anti-inflammatory agents from plant tissue comprises one or more of curcumin, resveratrol, pycnogenol and rosmarinic acid.
